(19) United States
(12) Patent Application Publication
(10) Pub. No.: US 2005/0234422 A1
(43) Pub. Date: Oct. 20, 2005

(54) Medicine bottle for injection

(76) Inventors: Jong Suk Oh, Gwangju (KR); Hyun Ju Oh, Gwangju (KR); Won Tai Oh, Gwangju (KR)

Correspondence Address: LAW OFFICES OF ROYA W. CRAIG, Suite 153, 10 N. Calvert St., Baltimore, MD, 21202, US

(21) Appl. No.: 11/030,736

(22) Filed: Jan. 6, 2005

(30) Foreign Application Priority Data
Apr. 19, 2004 (KR) 20-2004-23739
Nov. 4, 2004 (KR) 10-2004-89094

(51) Int. Cl. A61B 19/00; B65D 1/04; B65D 39/00

(52) U.S. Cl. 604/415; 215/DIG. 3; 215/6

(57) ABSTRACT

The present invention relates to a medicine bottle for injection capable of separately storing medicine powder and a solvent such as a saline solution in a single medicine bottle and conveniently mixing them therein in use. An inner wall 6 is provided to extend upwardly from the bottom of the medicine bottle therein such that the interior of a body 2 of the medicine bottle is partitioned by the inner wall into a portion 8 for storing the medicine powder and a portion 9 for storing the solvent but the portions communicate with each other at an upper end of the body 2.

### CROSS-REFERENCE TO RELATED APPLICATIONS

The present application derives priority from Korean Utility Model Apln. No.2004-23739 filed on Aug. 19, 2004, and Korean Patent Apln No. 2004-89094 filed on Nov. 4, 2004.

### TECHNICAL FIELD

The present invention relates to a medicine bottle for injection, and more particularly, to a medicine bottle for injection, wherein an inner wall is provided to partition the interior of a body of the medicine bottle into a portion for storing medicine powder and a portion for storing a solvent so that the medicine powder, i.e. a powdery medicine, and the solvent such as a saline solution are separately stored in one medicine bottle and both of them are mixed to be used as an injection in use, thereby conveniently storing the medicine powder and solvent and easily mixing them with each other.

### BACKGROUND ART

Generally, as for conventional medicines or injections prepared by mixing two or more medicines or solvents in use, the mixed medicines can exhibit full efficacy only when they are used or injected just after the mixing thereof. If a certain period of time elapses after mixing, or the mixed medicines me stored at an improper temperature, the medicines cannot sufficiently exhibit their efficacy due to degeneration of the mixed medicines.

Further, upon storage of a medicine in a liquid state, there is a high possibility that the medicine may degenerate due to its storage temperature and the like. Thus, the shelf life of the medicine is shortened. It is preferred that a medicine be stored in a powder state and then mixed with a solvent such as a saline solution to be used as an injection. Therefore, a conventional medicine for injection is subjected to six steps: storing medicine powder for a liquid injection and separately storing a solvent in an additional container, extracting the solvent such as a saline solution from the container, inputting the solvent into a medicine bottle containing the medicine powder using a syringe, withdrawing the syringe rearward, shaking the medicine bottle to dissolve the medicine powder in the solvent, and extracting the mixed liquid medicine again and using the medicine as an injection.

In such steps, there is inconvenience in that two separate medicine bottles are required for separately storing the medicine powder and the solvent and they should be stored separately and carried with special attention upon treatment of a patient. Further, since the needle of the syringe should be inserted into and withdrawn from the powdery medicine container and the solvent storing container at least three times before the medicine injection is loaded into the syringe, there is a high probability that the needle of the syringe may be contaminated or may injure a user. Moreover, there is a case where upon loading of a solvent into a bottle using a syringe with the solvent contained therein, the solvent is not easily input into the bottle due to inner pressure of the bottle and the solvent flows back toward the syringe. Thus, the process of mixing medicine powder and a solvent, which are contained in separate medicine bottles, with each other has many problems as described above.

### DISCLOSURE OF INVENTION

The present invention is conceived to solve the problems in the prior art. An object of the present invention is to minimize inconvenience and the possibility of contamination involved in mixing medicine powder and a solvent with each other by separately storing the medicine powder and the solvent such as a saline solution in a single medicine bottle and by simply mixing the medicine powder and the solvent within the medicine bottle in use without utilizing a syringe to obtain an usable injection medicine.

According to the present invention for achieving the object, there is provided a medicine bottle for injection, comprising an inner wall for partitioning the interior of a body of the medicine bottle into a portion for storing medicine powder and a portion for storing a solvent. The inner wall may extend from a bottom of the body of the medicine bottle to a level below an upper end of the body so that the medicine powder storing portion and the solvent storing portion communicate with each other through the upper end of the body where the inner wall does not exist. Further, the inner wall may extend from a bottom of the body to a mouth of the body and a hole may be formed at an upper portion of the inner wall so that the medicine powder storing portion and the solvent storing portion communicate with each other therethrough. The medicine bottle with the hole may further comprise a rubber wall for hermetically closing a space where the inner wall does not exist between the mouth of the body and a level below the hole.

According to another feature of the present invention, there is provided a medicine bottle for injection, comprising an inner wall extending upwardly from a bottom of a body of the medicine bottle to partition the interior of the body into a portion for storing medicine powder and a portion for storing a solvent, and a rubber wall extending downwardly from an inner surface of a rubber stopper of a closure for the medicine bottle, whereby an upper surface of the inner wall and a lower surface of the rubber wall have portions in contact with each other, respectively. Here, the inner wall may be configured to longitudinally extend with a width corresponding to the diameter of the body at the center of the bottom thereof, and the rubber wall may be configured to longitudinally extend with a width corresponding to the diameter of the body at the center of the inner surface of the rubber stopper. The contact portions of the upper surface of the inner wall and the lower surface of the rubber wall may take the shape of “U” or “∩”, or have a rugged structure (sawtooth structure). In the medicine bottle of the present invention, the rubber wall with a cross section corresponding to half of the entire area of the rubber stopper may extend downwardly from the inner surface of the rubber stopper so that the upper surface of the inner wall can be in contact with the lower surface of the rubber wall, thereby fully closing either the medicine powder storing portion or the solvent storing portion.

According to a further feature of the present invention, there is provided a medicine bottle for injection, comprising an inner wall extending upwardly from a bottom of a body of the medicine bottle to partition the interior of the body into a portion for storing medicine powder and a portion for storing a solvent, and a rubber wall extending downwardly from an inner surface of a rubber stopper of a closure for the medicine bottle, whereby an upper surface of the inner wall and a lower surface of the rubber wall have portions in contact with each other, respectively, wherein the rubber wall is connected integrally with and extends downwardly from the inner surface of the rubber stopper such that as the rubber stopper is turned, the contact portions of the lower surface of the rubber wall and the upper surface of the inner wall are out of contact with each other and the rubber wall is then rotated to be out of joint with the inner wall. A protrusion may be provided on an inner surface of the body of the medicine bottle at a position angularly spaced apart from the inner wall by (90×odd number) degrees so that the rubber wall can be rotated through an angle of (90×odd number) degrees.

According to a still further feature of the present invention, the rubber stopper of the closure for the medicine bottle may be constructed such that a side surface of an aluminum cap fixed to the rubber stopper suspends below a mouth flange of the medicine bottle, an outer portion of a bottom surface of the aluminum cap bulges out to be spaced apart from the mouth flange and an inner portion of the bottom surface is attached to an inner portion of the bottom of the mouth flange, whereby when the aluminum cap is pulled upwardly, both the aluminum cap and the rubber stopper can be moved upwardly. Further, with the elasticity of the rubber material, the close contact of the lower surface of the rubber wall with the upper surface of the inner wall establishes a sealing structure. However, upon rotation of the rubber wall, they are easily out of contact with each other due to the elasticity of the rubber material and then rotated. Here, the rubber wall may be a rubber wall having superior elasticity so that the rubber wall can be directly rotated without pulling the rubber stopper upwardly.

According to a still further feature of the present invention, there is provided a medicine bottle for injection, comprising an inner wall extending from a bottom of the body to a mouth of the body and having a hole formed at an upper portion thereof so that a portion for storing medicine powder and a portion for storing a solvent communicate with each other therethrough, a rubber wall for hermetically closing a space where the inner wall does not exist between a mouth of the body and a level below the hole, and an aluminum cap and a rubber stopper constructed such that a side surface of the aluminum cap fixed to the rubber stopper suspends below a mouth flange of the medicine bottle, an outer portion of a bottom surface of the aluminum cap bulges out to be spaced apart from the mouth flange and an inner portion of the bottom surface is attached to an inner portion of the bottom of the mouth flange, whereby when the aluminum cap are pulled upwardly, both the aluminum cap and the rubber stopper can be moved upwardly and the hole is opened.

Further, in the medicine bottle of the present invention, the rubber stopper or the aluminum cap of a closure for the medicine bottle may be provided with a syringe needle penetrating portion at a position on an upper surface thereof that is opposite to a position on a lower surface thereof where the rubber wall does not exist.

Meanwhile, medicine bottles for injection made of any materials so far as they do not depart from the technical spirit of the present invention can fall within the scope of the present invention. Although the present invention relates to a medicine bottle for injection, it will be apparent that the medicine bottle can be implemented as any containers including containers for storing medicines, chemicals, microorganisms or microbiological agents, which may be broken or degenerated in a state dissolved in a saline solution, distilled water or other solvents, without modifying the technical features of the present invention.

Further, a medicine bottle with the technical features of the present invention may be a medicine bottle for injection that has two or more inner walls and rubber walls to establish one or more portions for storing other solid powder or solvents in addition to the medicine powder storing portion and the solvent storing portion within the body of the medicine bottle.

As described above, the present invention provides a medicine bottle in which a mouth of the bottle is closed by a closure consisting of a rubber stopper and an aluminum cap and an inner wall extending upwardly from a bottom of the bottle only to a level below an upper end of the body of the bottle is provided to separately store medicine powder and a solvent. A rubber wall extending downwardly from the rubber stopper at the mouth of the bottle comes into contact with the inner wall, thereby separately storing the medicine powder and the solvent in a hermetically sealed state. When an injection is intended to be used, the rubber stopper is turned so that the rubber wall can be rotated. At this time, the partitioned portions are caused to communicate with each other at the upper end of the body. Upon shaking of the bottle, the medicine powder and the solvent are mixed with each other.

### BRIEF DESCRIPTION OF DRAWINGS

FIG. 1 is a perspective view showing the basic structure of a medicine bottle for injection according to the present invention.

FIG. 2 is a perspective view showing another configuration of the medicine bottle for injection according to the present invention.

FIG. 3 is a perspective view showing an example of the medicine bottle for injection according to the present invention.

FIG. 4 is a sectional view showing the example of the medicine bottle for injection according to the present invention.

FIG. 5 is a sectional view showing another example of the medicine bottle for injection according to the present invention.

FIG. 6 is a sectional view showing a further example of the medicine bottle for injection according to the present invention.

FIG. 7 is a sectional view showing a state where a rubber stopper and an aluminum cap of the medicine bottle of the present invention are raised.

FIG. 8 is a sectional view showing a state where the rubber stopper and the aluminum cap of the medicine bottle of the present invention are raised and then turned.

FIG. 9 is a sectional view showing a state where the rubber stopper and the aluminum cap of the medicine bottle of the present invention are directly turned as they are.

FIG. 10 is a perspective view showing a state where the rubber stopper and the aluminum cap of the medicine bottle of the present invention are raised.

FIG. 11 is a sectional view showing a state where the medicine bottle of the present invention is inverted to take out an injection therefrom using a syringe.

### BEST MODE FOR CARRYING OUT THE INVENTION

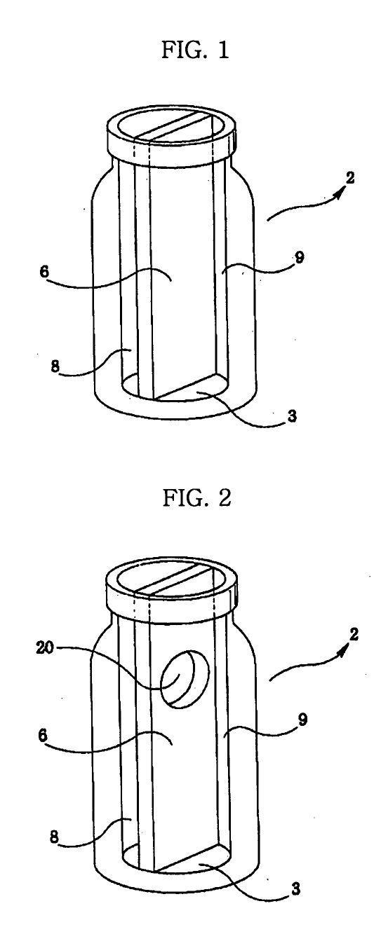

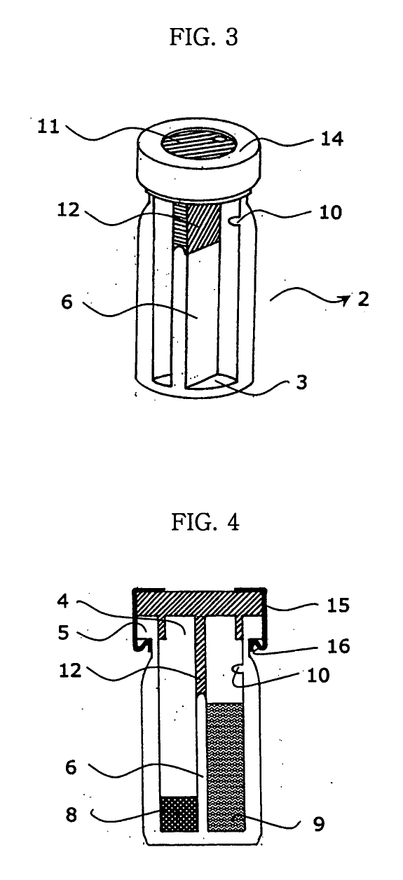

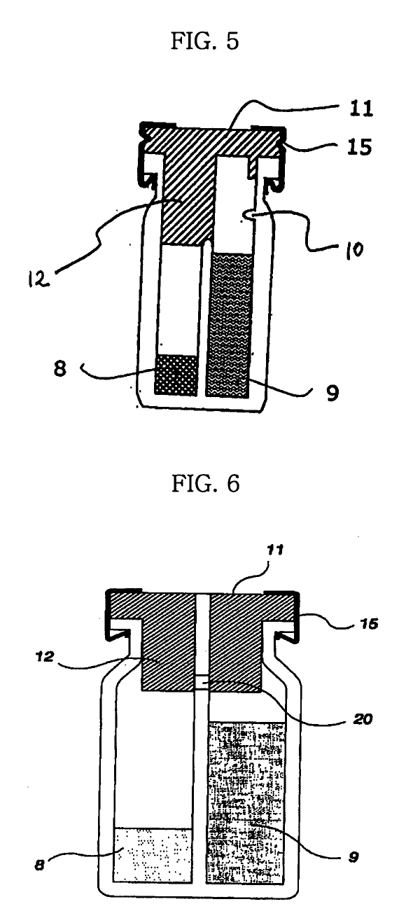

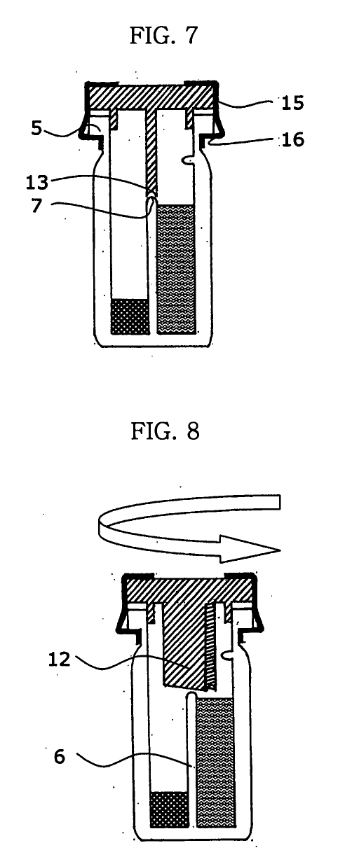

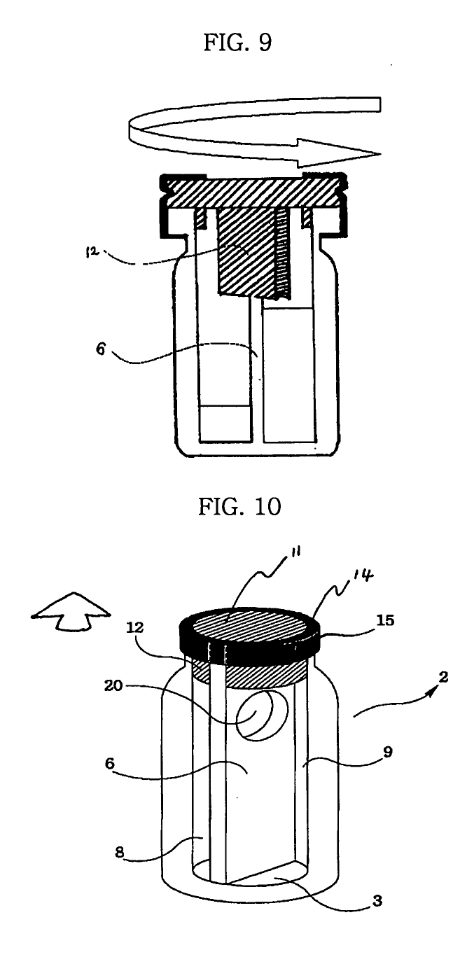

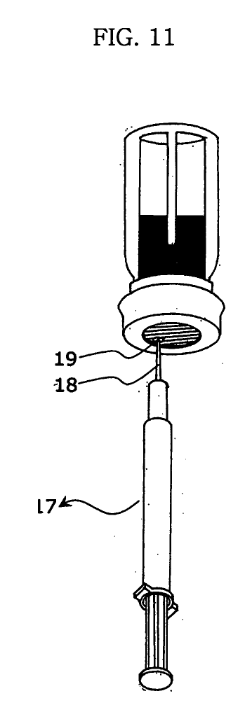

Hereinafter, the present invention will be described in detail with reference to accompanying drawings, in which FIG. 1 is a perspective view showing the basic structure of a medicine bottle for injection according to the present invention, FIG. 2 is a perspective view showing another configuration of the medicine bottle for injection according to the present invention, FIG. 3 is a perspective view showing an example of the medicine bottle for injection according to the present invention, FIG. 4 is a sectional view showing the example of the medicine bottle for injection according to the present invention, FIG. 5 is a sectional view showing another example of the medicine bottle for injection according to the present invention, FIG. 6 is a sectional view showing a further example of the medicine bottle for injection according to the present invention, FIG. 7 is a sectional view showing a state where a rubber stopper and an aluminum cap constituting a closure for the medicine bottle of the present invention are raised, FIG. 8 is a sectional view showing a state where the rubber stopper and the aluminum cap of the medicine bottle of the present invention are raised and then turned, FIG. 9 is a sectional view showing a state where the rubber stopper and the aluminum cap of the medicine bottle of the present invention are directly turned as they are, FIG. 10 is a perspective view showing a state where the rubber stopper and the aluminum cap of the medicine bottle of the present invention are raised, and FIG. 11 is a sectional view showing a state where the medicine bottle of the present invention is inverted to take out an injection therefrom using a syringe.

First of all, as shown in FIG. 1, the present invention is directed to a medicine bottle in which the interior of a body 2 of the medicine bottle is partitioned into a portion 8 for storing medicine powder and another portion 9 for storing a saline solution. To partition the interior of the medicine bottle into two spaces, the present invention is characterized by an inner wall 6. As shown in FIG. 1, the inner wall 6 may traverse a bottom 3 of the medicine bottle: In addition, it will be apparent that an inner wall 6 extending upwardly from inner side surfaces of the medicine bottle or from between the bottom 3 and the side surfaces falls within the scope of the present invention so far as it extends upwardly toward the top of the medicine bottle. The inner wall 6 for establishing two or more spaces within such a single medicine bottle in the present invention allows the storage of medicine powder and a solvent, which have been stored in separate containers in the prior art, in the single medicine bottle.

In the present invention, as shown in FIG. 3, the inner wall 6 extends from the bottom of the body 2 of the medicine bottle to a level below an upper end of the body 2. The medicine powder storing portion 8 and the solvent storing portion 9 communicate with each other through the upper end of the body where the inner wall 6 does not exist. The communication means that the medicine powder and the solvent can freely move toward the solvent storing space and the medicine powder storing space, respectively, through the space at the upper end where the inner wall 6 does not exist, since the inner wall 6 extending upwardly from the bottom, side surfaces or between the bottom and the side surfaces of the body of the medicine bottle reaches only a portion below the upper end. In addition to the extension of the inner wall only to the portion below the upper end of the medicine bottle, such a communication structure of a single medicine bottle according to the present invention may be a communication structure in which an inner wall extends from the bottom of the body 2 to a mouth of the body and a hole is formed at an upper portion of the inner wall 6 so that the medicine powder storing portion 8 and the solvent storing portion 9 communicate with each other therethrough as shown in FIG. 2.

Further, as shown in FIG. 3, the present invention contemplates a medicine bottle for injection, wherein an inner wall 6 for partitioning the interior of the body 2 into the medicine powder storing portion 8 and the solvent storing portion 9 extends upwardly from the bottom of the body 2 and a rubber wall 12 extends downwardly from an inner surface of a rubber stopper 11 for the medicine bottle so that an upper surface 7 of the inner wall comes into contact with a lower surface 13 of the rubber wall. The present invention provides a medicine bottle structure in which the contact of the upper surface 7 of the inner wall with the lower surface 13 of the rubber wall allows the medicine powder and the solvent to be hermetically and separately stored, and the materials can move in response to rotation of the rubber wall 12.

As described above, the inner wall extending upwardly from the bottom 3 of the body of the medicine bottle and the rubber wall 12 extending downwardly from the rubber stopper of the closure for the medicine bottle are to partition the interior of the medicine bottle into the two spaces. Although the inner wall 6 and the rubber wall 12 may be of any configurations or shapes for partitioning a space while occupying the space, it is most preferred that the inner wall 6 be configured to longitudinally extend with a width corresponding to the diameter of the body 2 at the center of the bottom thereof, and the rubber wall 12 be configured to longitudinally extend with a width corresponding to the diameter of the body at the center of the inner surface of the rubber stopper 11 for the medicine bottle. The contact portions of the upper surface 7 of the inner wall and the lower surface 13 of the rubber wall are characterized in that the contact portions take the shape of “∩” or “U” or have a rugged structure (sawtooth structure) to hermetically store the medicine powder and the solvent while preventing them from being mixed with each other upon storage of them in a separate state. The rubber wall 12 extending from the inner surface of the rubber stopper 11 that plugs up the mouth 4 of the medicine bottle partitions an upper space of the interior of the body 2. If the contact portions of the upper surface 7 of the inner wall and the lower surface 13 of the rubber wall are formed to have rugged structures, the rugged structures of the contact portions of the upper surface 7 of the inner wall and the lower surface 13 of the rubber wall improve sealing effects for the medicine powder storing portion 8 and the solvent storing portion 9. This allows the rubber wall and the inner wall 6 to be rotated and be out of joint or to be engaged with each other due to elasticity of the rubber wall 12 with the “U”-shaped or rugged structure when the rubber wall 12 is turned in the future.

Further, as shown in FIG. 5, another example of the medicine bottle for injection according to the present invention is a medicine bottle for injection characterized in that a rubber wall 12 with a cross section corresponding to half of the entire area of the rubber stopper 11 extends downwardly from the inner surface of the rubber stopper 11 so that the lower surface 13 of the rubber wall abuts the upper surface 7 of the inner wall, thereby fully closing either the medicine powder storing portion 8 or the solvent storing portion 9 at a time. In the medicine bottle manufactured such that the rubber wall 12 has the cross section corresponding to half of the entire area of the rubber stopper, when an aluminum cap 14 and the rubber stopper 11 are turned to a protrusion 10 which protrudes from an upper portion of a body 2 of the medicine bottle at a position perpendicular to the inner wall 6, the rubber wall 12 is out of joint with the inner wall 6 so that the medicine powder storing portion 8 and the solvent storing portion 9 can communicate with each other and the medicine and the solvent can be easily mixed with each other upon shaking of the medicine bottle.

Moreover, a further example of the medicine bottle for injection according to the present invention is a medicine bottle for injection, wherein an inner wall 6 extends from the bottom to the mouth of the body 2 and the medicine powder storing portion 8 and the solvent storing portion 9 communicate with each other through a hole formed at an upper portion of the inner wall 6. As shown in FIG. 6, the medicine bottle for injection is characterized in that the inner wall 6 formed with the hole extends to the upper end of the medicine bottle and a rubber wall is further provided to occupy and close spaces except the inner wall between the levels of the mouth of the body and a portion below the hole. The rubber wall 12 occupies and closes the both spaces partitioned by and except the inner wall 6 while extending to such an extent that it covers the hole through which the medicine powder storing portion 8 and the solvent storing portion 9 communicate with each other. Such a rubber wall 12 separately and hermetically closes the medicine powder storing portion 8 and the solvent storing portion 9 and can be simply raised to reveal the hole upon mixing the medicine powder and the solvent with each other. A syringe needle penetrating portion 19 of the rubber stopper is formed to be thin so that a syringe needle 18 can easily go therethrough.

When the medicine powder and the saline solution are intended to be mixed with each other, the aluminum cap 14 and the rubber stopper 11 are pulled upwardly at a mouth flange 5 of the medicine bottle so that the upper surface 7 of the inner wall is out of contact with the lower surface 13 of the rubber wall as shown in FIG. 7, and thus, the aluminum cap 14 and the rubber stopper 11 can be easily turned as shown in FIG. 8. An example of this preferred embodiment of the present invention is a medicine bottle for injection characterized in that the rubber wall 12 is connected integrally with and extends downwardly from the inner surface of the rubber stopper 11 for the medicine bottle, as shown in FIG. 7. The rubber wall 12 is constructed such that as the rubber stopper 11 is raised, the contact portions of the lower surface 13 of the rubber wall and the upper surface 7 of the inner wall are out of contact with each other and the rubber wall 12 is then rotated to be out of joint with the inner wall as shown in FIG. 8. The structure's of the rubber wall and the rubber stopper 11 of the medicine bottle according to the present invention enable the rubber wall 12 extending below the rubber stopper 11 to be rotated when the rubber stopper 11 is turned and to simultaneously communicate the medicine powder storing portion 8 and the solvent storing portion 9 with each other within the medicine bottle.

Here, although there is no limitation on the angle of rotation of the aluminum cap 14 and the rubber stopper 11, it is preferred that the angle defined between the inner wall and the rubber wall be 90 degrees to maximize the mixing of the medicine powder and the solvent. Thus, a protrusion 10 may be provided on an inner surface of the body of the medicine bottle at a position angularly spaced apart from the inner wall by (90×odd number) degrees so that the rubber wall can be rotated through an angle of (90×odd number) degrees.

As described above, the aluminum cap 14 and the rubber stopper 11 should be easily turned while the upper surface 7 of the inner wall is out of contact with the lower surface 13 of the rubber wall. When the rubber stopper 11 is turned in such a manner, the rubber wall 12 is also turned while being out of joint with the inner wall 6. Since the present invention is characterized in that medicine powder and a solvent are stored separately and then mixed within a single medicine bottle in use, the medicine contained in the bottle should not leak out from the bottle when they are mixed through shaking of the bottle.

To this end, as shown in FIGS. 4 and 6, the rubber stopper 11 of the closure for the medicine bottle of the present invention is characterized in that a side surface 15 of the aluminum cap fixed to the rubber stopper 11 suspends below the mouth flange 5 of the medicine bottle, an outer portion of a bottom surface 16 of the aluminum cap bulges out to be spaced apart from the mouth flange and an inner portion of the bottom surface 16 is attached to an inner portion of the bottom of the mouth flange, so that when the aluminum cap 14 are pulled upwardly, both the aluminum cap 14 and the rubber stopper 11 can be moved upwardly. When the medicine powder and the solvent are intended to be mixed with each other, as shown in FIGS. 7 and 8, the aluminum cap 14 is pulled upwardly by applying a force thereto. At this time, a lower portion of the side surface 15 of the aluminum cap is easily pulled upwardly so that both the aluminum cap 14 and the rubber stopper 11 can be moved above the mouth of the medicine bottle. However, since the inner portion of the bottom surface 16 of the aluminum cap is fixed to the bottom of the mouth flange 5, the aluminum cap 14 and the rubber stopper 11 do not come out from the mouth 4 of the medicine bottle.

Although a communication passage can be established within the medicine bottle by raising and turning the closure in its close state using such a structure of the aluminum cap, a medicine bottle according to another embodiment is constructed such that the closure can be directly turned as it is without raising the closure, as shown in FIG. 9. The elasticity of the rubber material enables the lower surface 13 of the rubber wall to be out of contact with the upper surface 7 of the inner wall so that the rubber wall can be turned and then be out of joint with the inner wall. With the elasticity of the rubber material, the close contact of the lower surface 13 of the rubber wall with the upper surface 7 of the inner wall establishes a sealing structure. However, upon rotation of the rubber wall, they are easily out of contact with each other due to the elasticity of the rubber material and then rotated. When the aluminum cap 14 is turned by hand or tool in order to mix the medicine powder and the solvent with each other, the contact portion of the lower surface 13 of the rubber wall with the “U”-shaped or rugged structure is out of contact with the contact portion of the upper surface 7 of the inner wall. In a case where contact surfaces of rubber and glass members are flat, if the rubber member is pushed laterally on the surface of the glass member, it can be easily slid thereon. However, if the contact surfaces of the rubber and glass members have a “U-”shaped or rugged structure, the elasticity of the rubber member ensures superior sealing effects in a normal closed state and enables the rubber member to be easily turned against the glass inner wall and thus out of joint with the glass inner wall by simply turning the closure if necessary. When the aluminum cap 14 is turned in the present invention, the rubber stopper 11 fixed to the aluminum cap 14 is also turned at the same time, as shown in FIG. 9. As the aluminum cap 14 and the rubber stopper 11 are turned, the rubber wall 12 is out of joint with the inner wall 6 so that the medicine powder storing portion 8 and the solvent storing portion 9 can communicate with each other. At this time, if the medicine bottle 1 is shaken, the medicine powder and the solvent are mixed with each other.

In the medicine bottle according to the further embodiment of the present invention, the medicine powder storing portion 8 and the solvent storing portion 9 can be caused to communicate with each other without raising the closure. The communication can be accomplished using the medicine bottle for injection shown in FIG. 6, wherein the inner wall 6 is formed with the hole at an upper portion thereof and the rubber wall 12 is further provided to occupy and close the spaces except the inner wall 6 between the levels of the mouth of the body and a portion below the hole 20. As can be seen in FIG. 10, in the medicine bottle manufactured to have the inner wall 6 formed with the hole 20 and the aluminum cap 14 that has the side surface 15 and the bottom surface 16 and covers the rubber stopper 11 filled with the rubber wall 12, the inner wall 6 and the rubber wall 12 separately and hermetically close the medicine powder storing portion 8 and the solvent storing portion 9. Upon mixing of the medicine powder and the solvent, the rubber stopper 11 and the rubber wall 12 are raised simply by pulling the aluminum cap 14 upwardly to reveal the hole.

Further, the rubber stopper 11 at the mouth 4 of the medicine bottle is secured by the aluminum cap 14. To cause the rubber stopper 11 to move together with the aluminum cap 14, the side surface 15 of the aluminum cap that comes into contact with the rubber stopper 11 is locally pressed on some portions thereof so that the pressed portions of the side surface of the aluminum cap can be stuck into the rubber stopper 11 or into a linear groove formed on a side surface of the rubber stopper 11, or the aluminum cap 14 is fixed to the rubber stopper 11 using an adhesive.

Since the present invention is directed to a medicine bottle for injection characterized in that the interior of a body of the medicine bottle is partitioned into two spaces, a rubber wall for partitioning the interior of the body is provided on an inner surface of a lid or closure for the medicine bottle. Accordingly, when an injection contained in the medicine bottle is extracted using a syringe, a needle of the syringe should enter the medicine bottle at a position on the closure where the rubber wall does not exist. Thus, a syringe needle penetrating portion should be provided at the position on the closure where the rubber wall does not exist. The syringe needle penetrating portion is provided at each of two or more spaces partitioned by the rubber wall. When an injection produced through the mixing of the medicine powder and the solvent is extracted using a syringe 17, a syringe needle 18 is caused to penetrate through the rubber stopper 11 at a position where the rubber wall 12 does not exist other than the center of the rubber stopper 11 formed with the rubber wall 12, the medicine bottle is inverted so that the mouth 4 of the medicine bottle is oriented downward, and the injection is then extracted, as shown in FIG. 11. At this time, since the injection is collected in both spaces on opposite sides of the rubber wall 12 traversing the inner surface of the rubber stopper 11, the syringe needle 18 should be caused to penetrate into the respective spaces on the opposite sides of the rubber wall 12 to extract the entire injection. Thus, in the case where the rubber wall 12 fully occupies one of the spaces as shown in FIG. 5, the syringe needle penetrating portion 19 through which the syringe needle 18 penetrates is provided on the rubber stopper 11 or the aluminum cap 14 within an area thereof unoccupied by the rubber wall. If the rubber wall 12 fully occupies one of the spaces, one glass protrusion 10 is formed to protrude from the inner surface of the medicine bottle. If the rubber wall does not fully occupy any one of the spaces, two glass protrusions may be formed.

Meanwhile, since the present invention is characterized in that the inner wall 6 is provided to partition the interior of the body 2 of the medicine bottle into the medicine powder storing portion 8 and the solvent storing portion 9, and the inner wall extends upwardly from the bottom of the body 2 and the rubber wall 12 extends downwardly from the inner surface of the rubber stopper 11 for the medicine bottle so that the lower surface 13 of the rubber wall abuts the upper surface 7 of the inner wall, the scope of the present invention intends to cover a medicine bottle for injection in which the body 2, the rubber stopper 11 and the aluminum cap 14 are made of any materials including glass, materials such as plastics originating from petroleum, or metals without departing from the scope of the technical features of the present invention. Although the present invention is directed to a medicine bottle for injection, it will be apparent that the medicine bottle can be implemented as any containers including containers for storing medicines, chemicals, microorganisms or microbiological agents, which may be broken or degenerated in a state dissolved in a saline solution, distilled water or other solvents, without modifying the technical features of the present invention.

Further, a medicine bottle with the technical features of the present invention may be a medicine bottle for injection that has two or more inner walls 6 and rubber walls to establish one or more portions for storing other solid powder or solvents in addition to the medicine powder storing portion 8 and the solvent storing portion 9 within the body 2 of the medicine bottle.

### INDUSTRIAL APPLICABILITY

According to the present invention, medicine powder and a solvent such as a saline solution are separately stored in a single medicine bottle and then mixed with each other therein to produce an injection in use. Therefore, the medicine powder and the solvent can be accurately and conveniently mixed with each other, and it is possible to minimize the time delay in mixing the medicine powder and the solvent with each other, and the possibility of contamination thereof.

1. A medicine bottle for injection, comprising:
an inner wall (6) for partitioning the interior of a body (2) of the medicine bottle into a medicine powder storing portion (8) and a solvent storing portion (9).

2. The medicine bottle as claimed in claim 1, wherein the inner wall (6) extends from a bottom of the body (2) of the medicine bottle to a level below an upper end of the body (2) so that the medicine powder storing portion (8) and the solvent storing portion (9) communicate with each other through the upper end of the body where the inner wall (6) does not exist.

3. The medicine bottle as claimed in claim 1, wherein the inner wall (6) extends from a bottom of the body (2) to a mouth of the body and a hole is formed at an upper portion of the inner wall (6) so that the medicine powder storing portion (8) and the solvent storing portion (9) communicate with each other therethrough.

4. The medicine bottle as claimed in claim 3, further comprising a rubber wall for hermetically closing a space where the inner wall does not exist between the mouth of the body and a level below the hole.

5. A medicine bottle for injection, comprising:
an inner wall (6) extending upwardly from a bottom of a body (2) of the medicine bottle to partition the interior of the body (2) into a medicine powder storing portion (8) and a solvent storing portion (9), and
a rubber wall (12) extending downwardly from an inner surface of a rubber stopper (11) of a closure for the medicine bottle,
whereby an upper surface (7) of the inner wall and a lower surface (13) of the rubber wall have portions in contact with each other, respectively.

6. The medicine bottle as claimed in claim 5, wherein the inner wall (6) is configured to longitudinally extend with a width corresponding to the diameter of the body (2) at the center of the bottom thereof, and the rubber wall (12) is configured to longitudinally extend with a width corresponding to the diameter of the body at the center of the inner surface of the rubber stopper (11).

7. The medicine bottle as claimed in claim 5, wherein the contact portions of the upper surface (7) of the inner wall and the lower surface (13) of the rubber wall take the shape of “U” or “∩”.

8. The medicine bottle as claimed in claim 5, wherein the contact portions of the upper surface (7) of the inner wall and the lower surface (13) of the rubber wall have a rugged structure (sawtooth structure).

9. The medicine bottle as claimed in claim 5, wherein the rubber wall (12) with a cross section corresponding to half of the entire area of the rubber stopper (11) extends downwardly from the inner surface of the rubber stopper (11), thereby fully closing either the medicine powder storing portion (8) or the solvent storing portion (9) at a time.

10. The medicine bottle as claimed in claim 5, wherein the rubber wall (12) is connected integrally with and extends downwardly from the inner surface of the rubber stopper (11) such that as the rubber stopper (11) is turned, the contact portions of the lower surface (13) of the rubber wall and the upper surface (7) of the inner wall are out of contact with each other and the rubber wall (12) is then rotated to be out of joint with the inner wall.

11. The medicine bottle as claimed in claim 10, wherein a protrusion (10) is provided on an inner surface of the body of the medicine bottle at a position angularly spaced apart from the inner wall by (90×odd number) degrees so that the rubber wall can be rotated through an angle of (90×odd number) degrees.

12. The medicine bottle as claimed in claim 10, wherein the rubber stopper (11) of the closure for the medicine bottle is constructed such that a side surface (15) of an aluminum cap (14) fixed to the rubber stopper (11) suspends below a mouth flange (5) of the medicine bottle, an outer portion of a bottom surface (16) of the aluminum cap bulges out to be spaced apart from the mouth flange and an inner portion of the bottom surface (16) is attached to an inner portion of the bottom of the mouth flange, whereby when the aluminum cap (14) are pulled upwardly, both the aluminum cap (14) and the rubber stopper (11) can be moved upwardly.

13. The medicine bottle as claimed in claim 10, wherein the rubber wall (12) is a rubber wall (12) having superior elasticity so that the rubber wall can be directly rotated without pulling the rubber stopper (11) upwardly.

14. A medicine bottle for injection, comprising:
an inner wall (6) extending from a bottom of the body (2) to a mouth of the body and having a hole formed at an upper end thereof so that a medicine powder storing portion (8) and a solvent storing portion (9) communicate with each other therethrough,
a rubber wall for hermetically closing a space where the inner wall does not exist between a mouth of the body and a level below the hole, and
an aluminum cap (14) and a rubber stopper (11) constructed such that a side surface (15) of the aluminum cap (14) fixed to the rubber stopper (11) suspends below a mouth flange (5) of the medicine bottle, an outer portion of a bottom surface (16) of the aluminum cap bulges out to be spaced apart from the mouth flange and an inner portion of the bottom surface (16) is attached to an inner portion of the bottom of the mouth flange, whereby when the aluminum cap (14) are pulled upwardly, both the aluminum cap (14) and the rubber stopper (11) can be moved upwardly and the hole is opened.

15. The medicine bottle as claimed in claim 10 or 12, wherein the rubber stopper (11) of a closure for the medicine bottle is provided with a syringe needle penetrating portion (19) at a position on an upper surface thereof that is opposite to a position on a lower surface of the rubber stopper (11) where the rubber wall (12) does not exist.

16. The medicine bottle as claimed in claim 5, 10, 12 or 14, wherein the body (2), the rubber stopper (11) and the aluminum cap (14) are made of glass, materials including plastics originated from petroleum, or metals.

17. The medicine bottle as claimed in claim 1, 5, 10, 12 or 14, wherein the medicine bottle is a container for storing a medicine, a chemical, a microorganism or a microbiological agent which may be broken or degenerated in a state dissolved in a saline solution, distilled water or other solvents.

18. The medicine bottle as claimed in claim 1, 5, 10, 12 or 14, wherein two or more inner walls (6) are provided to establish one or more portions for storing other solid powder or solvents in addition to the medicine powder storing portion (8) and the solvent storing portion (9) within the body (2) of the medicine bottle.
